(19) United States
(12) Patent Application Publication
(10) Pub. No.: US 2011/0137035 A1
(43) Pub. Date: Jun. 9, 2011

(54) PREPARATION OF MICROBIOLOGICALLY PRODUCED ERGOT ALKALOIDS

(75) Inventor: Stephan Bertel, Kundl (AT)

(73) Assignee: SANDOZ AG, Basel (CH)

(21) Appl. No.: 13/001,876

(22) PCT Filed: Jun. 26, 2009
(86) PCT No.: PCT/EP2009/058034

§ 371 Date: Jan. 24, 2011

(30) Foreign Application Priority Data
Jun. 30, 2008 (EP) 08159330.3

(51) Int. Cl. C07D 457/04

(52) U.S. Cl. 546/69

(57) ABSTRACT

The present invention relates to a method for preparing microbiologically produced ergot alkaloids of the following formula (I), comprising the step of: a) extracting the fermentation product occurring during the biological production, said product containing at least one ergot alkaloid of formula (I), at a pH of 8-14 using an extractant with a solubility in water of 0.2 g/100 g of water to 25 g/100 g water at 20° C., wherein the amount of extractant is sufficient to form a 2-phase system together with the fermentation product.

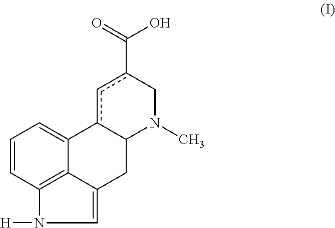

The present invention relates to processes for processing microbiologically produced ergot alkaloids of the formula I shown below

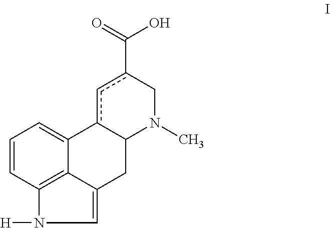

comprising the step:

a) extraction at a pH of 8-14 of the fermentation product obtained in the biological production, which contains at least one ergot alkaloid of the formula I, with an extraction agent which has a solubility in water at 20° C. of from 0.2 g/100 g of water to 25 g/100 g of water, wherein the amount of the extraction agent is sufficient to form a 2-phase system together with the fermentation product.

It is known that the ergot alkaloid paspalic acid inter alia is an important starting compound for the preparation of a number of paspalic acid derivatives with valuable pharmacological properties.

It is likewise known that the ergot alkaloid lysergic acid is an intermediate product for the preparation of important medicaments, such as e.g. the oxytoxic agents ergobasine and methylergobasine or the serotonin antagonist 1-methyl-d-lysergic acid (+)-2′-butanolamide. Lysergic acid can be obtained inter alia by rearrangement of paspalic acid.

The preparation of paspalic acid or lysergic acid with the aid of microbiological processes has been known for a long time and is carried out on a large industrial scale. The preparation of paspalic acid with the aid of the fungus species Claviceps paspali or its mutants [deposited under number NRRL 3080 at the United States Department of Agriculture (Northern Utilization Research and Development Division), Peoria/Ill.] has thus already been described in German Offenlegungsschrift DOS 1442294.

From the microbiologically produced paspalic acid, lysergic acid can be obtained by rearrangement in the strongly alkaline range. According to German Offenlegungsschrift DOS 1620375, this is preferably already carried out by treatment of the fermentation filtrate, after removal of the biomass, with bases, such as e.g. dilute ammonia solution or solutions of alkali metal hydroxides, optionally with heating, or first only after processing and purification of the microbiologically produced paspalic acid in an alkaline medium, with heating. The isomerization can also be carried out on a 2-phase mixture using tetraalkylammonium hydroxide, preferably tetrabutyl-ammonium hydroxide (U.S. Pat. No. 6242603).

The known microbiological processes for the production of ergot alkaloids, in particular paspalic acid or the isomerization product thereof, lysergic acid, require involved processing processes, such as e.g. separation of the biomass from the fermentation filtrate and the associated further purification or decolorizing steps and complicated separation steps on the crude product to give the desired end product. This leads not only to an involved and therefore longer total processing time in order to obtain the desired metabolism product of the fermentation process, but also to losses in yield, a limited total throughput of the process and as a result considerable production costs.

The object of the present invention was therefore to provide a process for processing microbiologically produced ergot alkaloids, in particular paspalic acid and lysergic acid, which brings about an improvement with respect to one or more of the abovementioned disadvantages of the prior art.

This object is achieved by providing the process according to the invention for processing microbiologically produced ergot alkaloids of the formula I shown below

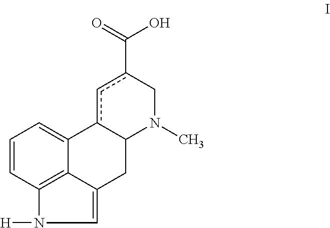

comprising the step:

a) extraction at a pH of 8-14 of the fermentation product obtained in the biological production, which contains at least one ergot alkaloid of the formula I, with an extraction agent which has a solubility in water at 20° C. of from 0.2 g/100 g of water to 25 g/100 g of water, wherein the amount of the extraction agent is sufficient to form a 2-phase system together with the fermentation product.

It has been found in fact, surprisingly, that ergot alkaloids of the formula I can be extracted at an alkaline pH, such as from 8 to 14, with a moderately polar solvent. According to the invention, a moderately polar solvent is understood as meaning a solvent or extraction agent which is sufficiently apolar to still form 2-phase systems with water, but is sufficiently polar to dissolve the ergot alkaloids, which are negatively charged at an alkaline pH. Unexpectedly, it is in fact possible in this way to transfer the ergot alkaloids in the extraction system, which are negatively charged at an alkaline pH, from the aqueous fermentation product and the extraction agent according to the invention into the phase of the extraction agent which is less polar compared with the aqueous fermentation product, which was not to be predicted in this way because of the charge.

As already mentioned above, a specific strain of the species Claviceps paspali Stevens et Hall is preferably used for the microbiological production of the ergot alkaloids paspalic acid of the formula Ia

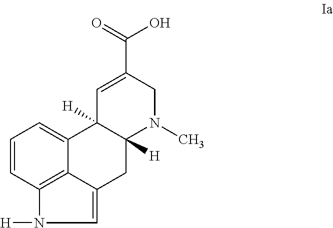

and lysergic acid of the formula Ib

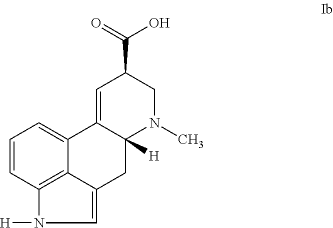

The samples of this strain are deposited at the United States Department of Agriculture (Northern Utilization Research and Development Division), Peoria/Ill. under number NRRL 3080. The corresponding culture conditions are disclosed inter alia in the German Offenlegungsschrift DOS 1442294, pages 5-11. The corresponding disclosure is introduced herewith as reference and is intended to be part of the disclosure of the present application.

For the processing according to the invention of the ergot alkaloids obtained in the microbiological process, in particular paspalic acid, the entire fermentation broth, without separation of the biomass from the fermentation filtrate, can be used as the fermentation product for further processing. This entire fermentation broth contains as ergot alkaloids essentially paspalic acid as the main metabolism product of the microbiological process, in addition to smaller amounts of lysergic acid and where appropriate lysergic acid derivatives. The entire fermentation broth conventionally has an ergot alkaloid titer of between 0.5-40 g/kg of entire fermentation broth. The fermentation filtrate can also be employed as the fermentation product used for the extraction as long as the additional outlay for separating off the biomass from the fermentation filtrate is not shied away from. Preferably, according to the invention the entire fermentation broth without separation of the biomass from the filtrate is employed as the fermentation product.

The fermentation product is extracted at least once with an extraction agent which has a solubility in water at 20° C. of from 0.2 g/100 g of water to 25 g/100 g of water. In this context, an extraction agent which is essentially free from halogenated hydrocarbons is preferred, this meaning according to the invention that halogenated hydrocarbons occur in the extraction agent with a content of less than 2% by volume, based on the total volume, and more preferably do not occur in the extraction agent.

Preferably, the fermentation product is extracted at least once with, as the extraction agent, a short-chain, aliphatic alcohol or ester which has at most a limited solubility in water. Preferably, the extraction is carried out several times, particularly preferably twice, preferably with the same extraction agent, the amount of extraction agent in each case being sufficient to form a 2-phase system together with the fermentation product. The person skilled in the art knows that in addition to the two liquid phases present side by side, solid particles from the fermentation process are also present in the abovementioned system.

Preferably, liquid aliphatic alcohols or esters with a solubility in water of at most 25 g/100 g of water (at 20° C.), preferably with a solubility in water of from 0.2 g/100 g of water to 25 g/100 g of water, particularly preferably with a solubility in water at 20° C. of from 0.5 g/100 g of water to 15 g/100 g of water, very particularly preferably 2 g/100 g of water to 10 g/100 g of water are employed as the extraction agent. Such extraction agents which have only a limited solubility in water are, preferably, aliphatic alcohols having at least 4 C atoms, particularly preferably alcohols having 4 to 7 C atoms, preferably n-butanol, isobutanol and/or sec-butyl alcohol and/or at least one aliphatic ester having at least 2 to 4 C atoms, preferably methyl acetate, ethyl formate or methyl formate. A mixture of two or more of the abovementioned alcohols, a mixture of two or more of the abovementioned esters or a mixture of in each case one or more of the abovementioned alcohols with in each case one or more of the abovementioned esters can also be employed for the extraction. Butanol, such as n-butanol and/or sec-butyl alcohol, and/or ethyl formate is very particularly preferably used.

By employing the extraction agents mentioned, the extraction of the ergot alkaloids according to formula I, in particular of paspalic acid and of the lysergic acid which is possibly present, surprisingly takes place very selectively if the pH of the fermentation product has been adjusted to an alkaline pH of between 8-14, preferably 9.5 to 11.5. Particularly preferably, the pH is adjusted to a value in the range of 10.0-11.0. The pH can be adjusted by addition of bases, such as e.g. alkali metal hydroxides, preferably sodium hydroxide solution or potassium hydroxide solution, or by introduction of bases, such as e.g. ammonia or tetra(C1-C6)alkylammonium hydroxide, preferably tetrabutylammonium hydroxide, optionally in a mixture with a smaller amount of alkali metal hydroxides. Preferably, during the particular extraction the fermentation product is agitated, such as e.g. shaken or stirred, together with the extraction agent. It is also possible to carry out the extraction step in the counter-current process, as a result of which an at least partly continuous processing is possible.

The suitable amount of extraction agent, which can also vary according to the extraction procedure, can be determined by simple preliminary experiments and is not limited, as long as sufficient extraction agent is used to form a 2-phase system with the fermentation product. As a rule, 0.2 to 5 volumes of extraction agent per volume of fermentation product can be used with good results.

The corresponding devices are known to the person skilled in the art, as are the extraction apparatuses employed. The extracts obtained by the extraction are typically combined.

The temperature for the extraction of the fermentation product is likewise not limited in particular, and the extraction can be carried out at room temperatures in a range of 20-25° C.

For further isolation of the ergot alkaloids of the formula I, the extract obtained can be concentrated by at least partly removing the extraction agent. A subsequent crystallization of the ergot alkaloid can thereby be facilitated. For this, preferably at least an amount of extraction agent is removed such that the concentration of ergot alkaloids of the formula I in the concentrate is doubled, preferably increased up to 4-fold. The concentration is preferably carried out under gentle conditions, that is to say, for example, at room temperature in vacuo. A moderate increase in temperature, in order to facilitate the concentration, up to 70 ° C. for example is entirely possible, especially in the preparation of lysergic acid.

Steps b) and c) can then be carried out in the further processing.

Thus, in step b) an aqueous solution is added to the extract which has been separated off from the remaining entire fermentation broth, in order to obtain a 2-phase mixture, and in step c) the pH of the 2-phase mixture obtained in b) is adjusted to a value of from 2.0 to 8.0, preferably to a value of from 4.0 to 6.5 and more preferably to a value which essentially corresponds to the isoelectric point of one of the ergot alkaloids according to formula I, i.e. about 5.6 for paspalic acid and lysergic acid.

As a result, the ergot alkaloids according to formula I pass from the organic phase into the aqueous phase. The acid pH is preferably adjusted by addition of a suitable amount of sulfuric acid, hydrochloric acid or glacial acetic acid.

From the 2-phase mixture obtained in this way, crude ergot alkaloid of the formula I crystallizes out of the aqueous phase, it being possible for the speed of crystallization to be accelerated by cooling the 2-phase mixture to temperatures down to 10° C.

The crude ergot alkaloid crystals can be obtained from the 2-phase mixture e.g. by simple centrifugation, without the aqueous phase into which the ergot alkaloids of the formula I have passed having to be isolated beforehand. After the ergot alkaloids have passed into the aqueous phase this can of course be separated off and the crude ergot alkaloid crystals can be obtained therefrom, if the additional outlay for the phase separation is desired.

If no isomerization of paspalic acid into lysergic acid is carried out during the processing process according to the invention, the main product of the processing process according to the invention is paspalic acid, paspalic acid being understood according to the invention as a mixture containing paspalic acid and lysergic acid in which the molar content of paspalic acid predominates over lysergic acid. Thus, surprisingly, paspalic acid is already obtained with a high total yield, preferably of more than 65%, after the first crystallization, the paspalic acid crystals already having a surprisingly high purity, preferably of more than 90%.

To further improve the purity of the paspalic acid and optionally to achieve a complete decoloration of the product, the paspalic acid crystals isolated in this way can be dissolved again in suitable solvents and optionally treated with active charcoal in order to achieve complete decoloration and in order subsequently, by renewed acidification of the product-containing solution, preferably to the isoelectric point of paspalic acid of pH 5.6, to crystallize this out of the aqueous, acid solution and then to dry the paspalic acid crystals at conventional temperatures, preferably after a purification by washing with aqueous methanol solution. This achieves an increase in the purity of the paspalic acid crystals obtained in this way to more than 95% at a practically unchanged yield of paspalic acid of more than 65%.

If lysergic acid is to be obtained as the main product with the aid of the processing process according to the invention, the process additionally comprises a further step in which the pH of a solution or suspension containing paspalic acid is adjusted to a value of ≧11 to 14 and the paspalic acid is isomerized (rearranged) to lysergic acid, optionally with heating, lysergic acid being understood according to the invention as meaning a mixture containing paspalic acid and lysergic acid in which the molar content of lysergic acid predominates over the paspalic acid. Preferably, lysergic acid is to be understood according to the invention as meaning a mixture containing paspalic acid and lysergic acid in which the molar ratio of lysergic acid to paspalic acid is >4.

For the rearrangement, there are in principle at least three preferred possibilities for obtaining lysergic acid as the main product by isomerization (rearrangement) from the microbiologically produced ergot alkaloids of the formula I:
1. It is thus possible in principle to adjust the entire fermentation broth to preferably a pH of from 11 to 13 before the abovementioned step a), the extraction. This adjustment of the pH can preferably be carried out by introduction of ammonia, or addition of alkali metal hydroxides, preferably sodium hydroxide solution or potassium hydroxide solution, and/or tetraalkylammonium hydroxide, preferably tetra(C1-C6)alkylammonium hydroxide, particularly preferably tetrabutyl-ammonium hydroxide.

An increase in the reaction temperature, preferably to 30-70° C., particularly preferably to 50° C.-70° C., makes it possible to shorten the duration of the isomerization.

If an increase in the reaction temperature is carried out, it is expedient to carry out cooling to room temperature, preferably to 20-25° C., before step a), the extraction of the fermentation product, preferably of the entire fermentation broth, with at least one of the abovementioned extraction agents. The fermentation product should moreover be adjusted to a pH corresponding to that stated above for the extraction in step a).

The course of the rearrangement reaction can be monitored by in-process control with HPLC with the aid of the increase in lysergic acid according to formula Ib and the decrease in paspalic acid according to formula Ia. Typically, 4 h at pH 12.5 and 50° C. is sufficient to effect an essentially complete conversion into lysergic acid according to formula Ib, lysergic acid according to formula Ib being understood as meaning pure lysergic acid.

Further processing of the lysergic acid obtained in this way corresponds to the abovementioned process steps comprising steps a), b) and c) for processing ergot alkaloids of the formula I, including a recrystallization to increase the purity of the end product.
2. Alternatively, however, it is also possible only first to rearrange the paspalic acid isolated by the process according to the invention, optionally after the recrystallization step, into lysergic acid by dissolving the paspalic acid crystals in a corresponding alkaline medium, such as in an alkali metal hydroxide solution, preferably a sodium or potassium hydroxide solution, it being necessary to adjust the pH of the solution to a value of from 11 to 14 in order to rearrange the paspalic acid into lysergic acid, optionally with heating to 30-70° C.

As already stated above, the lysergic acid can be crystallized out of the alkaline medium by acidification with the abovementioned acids to a pH of from 2 to 8, preferably to the isoelectric point of lysergic acid. The corresponding crude crystals can also be recrystallized, as stated above, and finally dried by the conventional method and manner.
3. The step of alkali-catalyzed rearrangement of paspalic acid to lysergic acid can of course also be carried out in connection with other process steps in the sequence of the processing process according to the invention, that is to say e.g. only first in the extract obtained after the extraction of the fermentation product. Since lysergic acid does not differ decisively from paspalic acid with respect to its solubility properties, further processing can be carried out in accordance with the procedure comprising steps b) and c).

Since the extraction agents employed according to the invention for the extraction step a) are surprisingly very selective, the invention also provides the use of an extraction agent which has a solubility in water at 20° C. of from 0.2 g/100 g of water to 25 g/100 g of water for the extraction at a pH of 8-14 of ergot alkaloids of the formula I from an aqueous solution or suspension which contains the ergot alkaloids of the formula I.

Preferably, the abovementioned extraction agents which are employed in step a) of the processing process according to the invention are used for this extraction, particularly preferably alcohols having 4 to 7 C atoms and/or aliphatic esters having at least 2 to 4 C atoms. Butanol, such as n-butanol and/or sec-butyl alcohol, and/or ethyl formate is very particularly preferably used.

In spite of the purification process, these extraction agents employed can be detected in the processed ergot alkaloid of the formula I, e.g. by the “Method for determination of 1-butanol in ergot alkaloids of the formula I” described in the experimental part, which a person skilled in the art can adapt for other extraction agents in a suitable manner.

The present invention therefore also provides the products mentioned in the following:

Paspalic acid, preferably paspalic acid crystals, which has or have a content of from 2 ppm to 1,000 ppm, preferably 5 ppm to 700 ppm of in each case an extraction agent which has been employed in step a), preferably n-butanol and/or sec-butyl alcohol and/or ethyl formate;

Lysergic acid, preferably lysergic acid crystals, which has or have a content of from 2 ppm to 1,000 ppm, preferably 5 ppm to 700 ppm of in each case an extraction agent which has been employed in step a), preferably n-butanol and/or sec-butyl alcohol and/or ethyl formate.

Paspalic acid, preferably in the form of crystals, obtainable by a process according to the invention, in which the paspalic acid or crystals thereof has or have a content of 10 ppm to 500 ppm of in each case an extraction agent which has been employed in step a), preferably n-butanol and/or sec-butyl alcohol and/or ethyl formate, is furthermore preferred.

Lysergic acid, preferably in the form of crystals, obtainable by a process according to the invention, in which the lysergic acid or crystals thereof has or have a content of 10 ppm to 500 ppm of in each case an extraction agent which has been employed in step a), preferably n-butanol and/or sec-butyl alcohol and/or ethyl formate, is likewise furthermore preferred.

The invention also provides:
1. A process for processing microbiologically produced ergot alkaloids of the formula I

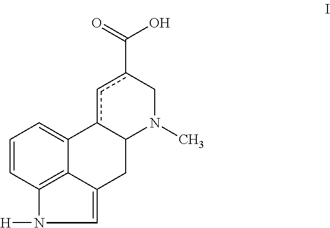

comprising the step:
   a) extraction at a pH of 8-14 of the fermentation product obtained in the biological production, which contains at least one ergot alkaloid of the formula I, with an extraction agent which has a solubility in water at 20° C. of from 0.2 g/100 g of water to 25 g/100 g of water, wherein the amount of the extraction agent is sufficient to form a 2-phase system together with the fermentation product.
2. A process according to item 1, characterized in that the extraction agent essentially comprises an alcohol having 4-7 carbon atoms and/or an ester having 2-4 carbon atoms.
3. A process according to item 1 or 2, characterized in that the extraction agent essentially comprises an alcohol chosen from n-butanol, n-pentanol, sec-butanol and isobutanol or essentially comprises an ester chosen from methyl acetate, methyl formate and ethyl formate or essentially comprises either a mixture of two or more of the abovementioned alcohols, a mixture of two or more of the abovementioned esters or a mixture of in each case one or more of the abovementioned alcohols with in each case one or more of the abovementioned esters.
4. A process according to one of items 1-3, characterized in that the extraction agent has a solubility in water at 20° C. of from 0.5 g/100 g of water to 15 g/100 g of water, preferably from 2 g/100 g of water to 10 g/100 g of water.
5. A process according to one of items 1-4, characterized in that the pH of the fermentation product is adjusted to a value in the range of 9.5-11.5, preferably from 10.0 to 11.0, before the extraction.
6. A process according to one of items 1-5, comprising the further steps:
   b) addition of an aqueous solution to the extract which has been separated off from the fermentation product, in order to obtain a 2-phase mixture;
   c) adjustment of the pH of the 2-phase mixture obtained in b) to a value of 2-8, in order to allow the ergot alkaloid of the formula I to crystallize out as the crude product.
7. A process according to one of items 1-6, characterized in that the crude product of the ergot alkaloids of the formula I is fed to further purification steps, in order to obtain ergot alkaloids of the formula I which are suitable for pharmaceutical purposes.
8. A process according to one of items 1-7, characterized in that the ergot alkaloid is paspalic acid.
9. A process according to one of the items 1-7, characterized in that the ergot alkaloid is lysergic acid and the process additionally comprises a step in which the pH of a solution or suspension containing paspalic acid is adjusted to a pH of from ≧11 to 14 and the paspalic acid is isomerized into lysergic acid, optionally with heating.
10. A process according to item 9, characterized in that the pH is adjusted to a value of from 11 to 13 by introduction of ammonia, or addition of alkali metal hydroxides, preferably sodium hydroxide solution or potassium hydroxide solution, and/or tetraalkylammonium hydroxide, preferably tetra(C1-C6)alkylammonium hydroxide, in order to effect the isomerization of paspalic acid to lysergic acid.
11. A process according to item 10, characterized in that the isomerization is carried out at a temperature of between 30° C. and 70° C.
12. A process according to one of items 9-11, characterized in that the isomerization of paspalic acid to lysergic acid takes place in the fermentation product before the extraction step a).
13. A process according to one of items 9-11, characterized in that the isomerization of paspalic acid to lysergic acid is carried out after the crude, crystallized paspalic acid has been obtained.
14. Use of an extraction agent which has a solubility in water at 20° C. of from 0.2 g/100 g of water to 25 g/100 g of water for the extraction at a pH of 8-14 of ergot alkaloids of the formula I from an aqueous solution or suspension which contains ergot alkaloids of the formula I.
15. Use according to item 14, characterized in that the extraction agent has a solubility in water of from 0.5 g/100 g of water to 15 g/100 g of water, preferably 2 g/100 g of water to 10 g/100 g of water.
16. Use according to item 14 or 15, characterized in that the ergot alkaloids of the formula I are paspalic acid of the formula Ia and/or lysergic acid of the formula Ib.
17. Paspalic acid, preferably paspalic acid crystals, which has a content of from 2 ppm to 1,000 ppm, preferably 5 ppm to 700 ppm of an extraction agent chosen from n-butanol, sec-butyl alcohol and ethyl formate.
18. Paspalic acid according to item 17, obtainable by a process according to one of items 1-10.
19. Lysergic acid, preferably lysergic acid crystals, which has a content of from 2 ppm to 1,000 ppm, preferably 5 ppm to 700 ppm of an extraction agent chosen from n-butanol, sec-butyl alcohol and ethyl formate.
20. Lysergic acid according to item 19, obtainable by a process according to one of items 9-13.

### EXAMPLE 1

1.25 kg of an entire fermentation broth which had a titer of 7.98 g of ergot alkaloid of the formula I/kg was adjusted to a pH of 10 by addition of NaOH and extracted twice with 1.25 l of n-butanol each time at this pH. The yield of ergot alkaloid of the formula I after the two extraction steps was 83%. The combined extracts were concentrated to about 30 g of ergot alkaloid of the formula I/kg of extract and about the same amount of water was added. Thereafter, the pH was adjusted to a value of 5.6 by addition of sulfuric acid. Paspalic acid passed from the organic into the aqueous phase and crystallized out. 7.2 g of paspalic acid with a purity of 93% were obtained in this way. This corresponds to a total yield of 67%.

### EXAMPLE 2

878 g of a fermentation broth which contained 2.1 g/kg of lysergic acid and 8.3 g/kg of paspalic acid were kept at pH 12.5 and 50° C. for 4 hours. After 4 hours, the broth contained 7.96 g/kg of lysergic acid and 0.37 g/kg of paspalic acid. The active compound was then extracted from the broth twice with 900 ml of n-BuOH each time and concentrated in vacuo to a content of 25 g/kg. Thereafter, the pH was adjusted to a value of 5.6 by addition of sulfuric acid, as a result of which the lysergic acid crystallized out.

The crude product obtained in this manner had a purity of 75%, and the yield, including the conversion, was 50%. By adding in the next cycle the butyl acetate mother liquor to the broth before the next conversion, it was possible to increase the yield to 70%.

### Comparison Process

Active charcoal is added to harvested broth containing paspalic and lysergic acid. At a neutral pH, paspalic and lysergic acid bind to the broth or to the active charcoal. The solid constituents are centrifuged off and the supernatant is discarded. The biomass isolated in this manner together with the active charcoal is then suspended with an ammoniacal alcohol solution, the two acids becoming detached from the solids. The two acids can finally be obtained in this way in the supernatant after a renewed centrifugation of the biomass and the active charcoal. When the supernatant is concentrated, paspalic and lysergic acid precipitate out as the crude product.

### Method for Determination of 1-Butanol in Ergot Alkaloids of the Formula I

The determination of 1-butanol in ergot alkaloids of the formula I was carried out by means of headspace

(HS) gas chromatography (GC) analysis. In this procedure, approx. 200 mg of ergot alkaloid sample were weighed into 20 ml HS vials and then taken up in 5 ml of 1-methylpyrrolidone.

Calibration was carried out with 8 points with approx. 1, 2, 4, 10, 25, 50, 100, 200 μg of 1-butanol in the HS vial, very small amounts of 1-butanol being detectable.

The headspace model G1888A from Agilent with a 1 ml sample loop was operated with the following parameters:

The sample was equilibrated at 80° C. on the “shaking high” setting for 30 min. The vial pressure of 15 psi was set for 0.2 min. The sample loop, heated to 200° C., was filled for 0.2 min with an equilibration time of 0.05 min. The injection time in the GC was 0.5 min; the transfer line in this procedure was at 200° C.

The injector block was heated to 230° C.; the split ratio was 5:1.

The column used was the type DB 624 from J&W with the serial number US8534615H. The column dimensions were 25 m length, 0.2 mm diameter and 1.12 μm film thickness of the stationary phase. The column was operated with a 1 ml constant flow of helium. The GC oven was operated with the following program: 40° C. was maintained for 0.5 min, heating was then carried out with a rate of 1° C./min to 45° C., then with 15° C./min to 180° C. and subsequently with 30° C./min to 220° C.; this temperature was maintained for 4.5 min.

A flame ionization detector (FID) was used as the detector. This was at 250° C. and was operated with 30 ml/min of hydrogen and 400 ml/min of synthetic air. The analog signal was transferred with the range 1 on the Chromeleon A/D converter.

1. Process for processing microbiologically produced ergot alkaloids of the formula I 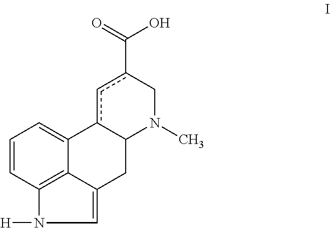
comprising the step:
a) extraction at a pH of 8-14 of the fermentation product obtained in the biological production, which contains at least one ergot alkaloid of the formula I, with an extraction agent which has a solubility in water at 20° C. of from 0.2 g/100 g of water to 25 g/100 g of water, wherein the amount of the extraction agent is sufficient to form a 2-phase system together with the fermentation product;
characterized in that the extraction agent essentially comprises an alcohol having 4-7 carbon atoms and/or an ester chosen from methyl acetate, methyl formate and ethyl formate.

2. Process for processing microbiologically produced ergot alkaloids of the formula I, comprising the step:
a) extraction at a pH of 8-14 of the fermentation product obtained in the biological production, which contains at least one ergot alkaloid of the formula I, with an extraction agent which has a solubility in water at 20° C. of from 0.2 g/100 g of water to 25 g/100 g of water, wherein the amount of the extraction agent is sufficient to form a 2-phase system together with the fermentation product;
characterized in that
i) a liquid aliphatic alcohol, in particular aliphatic alcohol having at least 4 C atoms, or
ii) a mixture of two or more liquid aliphatic alcohols, in particular aliphatic alcohols having at least 4 C atoms, or
iii) a mixture of in each case one or more liquid aliphatic alcohols, in particular aliphatic alcohols having at least 4 C atoms, and in each case one or more esters, in particular aliphatic esters having at least 2 to 4 C atoms,
is employed as the extraction agent.

3. Process according to claim 1 or 2, characterized in that the extraction agent essentially comprises an alcohol chosen from n-butanol, n-pentanol, sec-butanol and isobutanol or essentially comprises an ester chosen from methyl acetate, methyl formate and ethyl formate or essentially comprises either a mixture of two or more of the abovementioned alcohols, a mixture of two or more of the abovementioned esters or a mixture of in each case one or more of the abovementioned alcohols with in each case one or more of the abovementioned esters.

4. Process according to one of claims 1-3, characterized in that the pH of the fermentation product is adjusted to a value in the range of 9.5-11.5 before the extraction.

5. Process according to one of claims 1-4, comprising the further steps:
b) addition of an aqueous solution to the extract which has been separated off from the fermentation product, in order to obtain a 2-phase mixture;
c) adjustment of the pH of the 2-phase mixture obtained in b) to a value of 2-8, in order to allow the ergot alkaloid of the formula I to crystallize out as the crude product.

6. Process according to one of claims 1-5, characterized in that the ergot alkaloid is lysergic acid and the process additionally comprises a step in which the pH of a solution or suspension containing paspalic acid is adjusted to a pH of from ≧11 to 14 and the paspalic acid is isomerized into lysergic acid, optionally with heating.

7. Process according to one of claims 1-5, characterized in that the ergot alkaloid is paspalic acid of the formula Ia

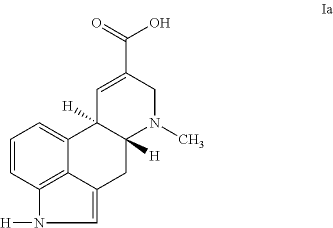

8. Use of an extraction agent which has a solubility in water at 20° C. of from 0.2 g/100 g of water to 25 g/100 g of water for the extraction at a pH of 8-14 of ergot alkaloids of the formula I from an aqueous solution or suspension which contains ergot alkaloids of the formula I, characterized in that the extraction agent essentially comprises an alcohol having 4-7 carbon atoms and/or an ester chosen from methyl acetate, methyl formate and ethyl formate.

9. Use of an extraction agent which has a solubility in water at 20° C. of from 0.2 g/100 g of water to 25 g/100 g of water for the extraction at a pH of 8-14 of ergot alkaloids of the formula I from an aqueous solution or suspension which contains ergot alkaloids of the formula I, characterized in that
i) a liquid aliphatic alcohol, in particular aliphatic alcohol having at least 4 C atoms, or
ii) a mixture of two or more liquid aliphatic alcohols, in particular aliphatic alcohols having at least 4 C atoms, or
iii) a mixture of in each case one or more liquid aliphatic alcohols, in particular aliphatic alcohols having at least 4 C atoms, and in each case one or more esters, in particular aliphatic esters having at least 2 to 4 C atoms,
is employed as the extraction agent.

10. Use according to claim 8 or 9, characterized in that the extraction agent has a solubility in water of from 0.5 g/100 g of water to 15 g/100 g of water, preferably 2 g/100 g of water to 10 g/100 g of water.

11. Use according to one of claims 8-10, characterized in that the ergot alkaloids of the formula I are
paspalic acid of the formula Ia 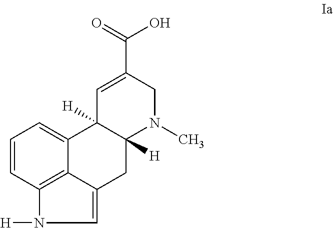
and/or lysergic acid of the formula Ib

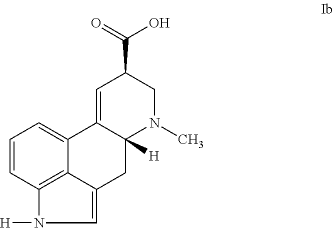

12. Paspalic acid, preferably paspalic acid crystals, obtainable by a process according to one of claims 1-5 and 7, which has a content of from 2 ppm to 1,000 ppm, preferably 5 ppm to 700 ppm of an extraction agent which was employed in step a).

13. Paspalic acid, preferably paspalic acid crystals, according to claim 12, which has a content of from 2 ppm to 1,000 ppm, preferably 5 ppm to 700 ppm of an extraction agent chosen from n-butanol, sec-butyl alcohol and ethyl formate.

14. Lysergic acid, preferably lysergic acid crystals, obtainable by a process according to one of claims 1-6, which has a content of from 2 ppm to 1,000 ppm, preferably 5 ppm to 700 ppm of an extraction agent which was employed in step a).

15. Lysergic acid, preferably lysergic acid crystals, according to claim 14, which has a content of from 2 ppm to 1,000 ppm, preferably 5 ppm to 700 ppm of an extraction agent chosen from n-butanol, sec-butyl alcohol and ethyl formate.
